(19) United States
(12) Patent Application Publication
(10) Pub. No.: US 2026/0325790 A1
(43) Pub. Date: Oct. 22, 2026

(54) DRUG DELIVERY SYSTEM FOR UPPER URINARY TRACT

(71) Applicants: THE UNIVERSITY OF CHICAGO, Chicago, IL (US); UNIVERSITY OF CINCINNATI, Cincinnati, OH (US)

(72) Inventors: Mohamed KAMEL, Cincinnati, OH (US); Omer RAHEEM, Chicago, IL (US)

(73) Assignees: THE UNIVERSITY OF CHICAGO, Chicago, IL (US); UNIVERSITY OF CINCINNATI, Cincinnati, OH (US)

(21) Appl. No.: 19/703,605

(22) Filed: Jun. 10, 2026

Related U.S. Application Data
(63) Continuation of application No. PCT/US2024/061460, filed on Dec. 20, 2024.
(60) Provisional application No. 63/613,341, filed on Dec. 21, 2023.

(51) Int. Cl. A61M 25/00; A61M 25/06; A61M 25/09; A61M 25/10

(52) U.S. Cl. CPC A61M 25/0041; A61M 25/0026; A61M 25/0097; A61M 25/007; A61M 2025/0681; A61M 25/09; A61M 2025/1015; A61M 2025/1052; A61M 2025/1061; A61M 2210/1082

(57) ABSTRACT

Systems and methods for delivering a drug to a patient's kidney. The present systems comprise a urethral catheter and a ureteral catheter sized to fit through the urethral catheter. The urethral catheter includes a balloon configured to be inflated in the patient's bladder to retain the urethral catheter in the patient's urethra. The ureteral catheter includes a balloon configured to be inflated in the patient's ureter and/or kidney while a distal end of the ureteral catheter is disposed in the patient's kidney. The ureteral catheter balloon can help retain liquid in the kidney to enable expanded contact areas with kidney tissues and extended dwell times (e.g., periods of exposure) of the drug in the kidney. The balloons also help retain the catheters in the patient's urinary tract, such that the catheter system can be used to deliver a drug to the patient's intermittently or periodically over a period of days or weeks.

### CROSS REFERENCE TO RELATED APPLICATIONS

This application is a continuation of International Application No. PCT/US2024/061460, filed Dec. 20, 2024, which claims priority of U.S. Provisional Application No. 63/613,341, filed Dec. 21, 2023, all of which applications are hereby incorporated by reference in their respective entireties.

### FIELD OF DISCLOSURE

The present system is generally related to urethral catheters and, more particularly but not by way of limitation, to systems and methods for urethral access to the upper urinary tract.

### BACKGROUND

Therapeutic drugs have conventionally been delivered to the kidneys through one of two approaches: (1) percutaneous antegrade approach or (2) transurethral retrograde approach.

In the percutaneous antegrade approach, an incision is formed through soft tissue of a patient's back overlying the patient's kidney, and a nephrostomy tube is passed through the incision and into the kidney. Drugs can thereafter be delivered to the kidney through the nephrostomy tube. The nephrostomy tube can be placed under sedation rather than general anesthesia, and can remain in place for several weeks to allow multiple drug administrations over time.

In the transurethral retrograde approach, with the patient in a supine position with legs elevated, a conventional ureteral catheter is inserted with the aid of a cytoscope through the patient's urethra and a ureter into a corresponding one of the patient's kidney.

### SUMMARY

Administering and retaining drugs to the interior of a patient's kidney can be challenging for a several reasons. For example, human kidneys are situated relatively deep in the abdomen, which can make it challenging to access via the natural urine exit path, the urethra. Additionally, urine typically transits through the kidneys to the bladder relatively quickly, meaning drugs delivered to the kidney will typically only stay in the kidney for a short period of time before being carried to the bladder by urine, thereby reducing the duration of contact of the drug with the urothelium (the inner lining of the kidney) and limiting the potential therapeutic effect. Additionally, the interior of the kidney is divided into compartments (calyces), which can make it difficult to deliver drug to all of the compartments or calyces, limiting the surface area and regions that may be contacted or treated by the drug.

Conventional approaches to delivering drugs to a patient's kidney suffer from a number of shortcomings that the present systems address. For example, while the percutaneous antegrade approach can be performed under sedation rather than general anesthetic and the nephrostomy tube can stay in place for several weeks, the percutaneous antegrade approach may suffer from (a) short durations or dwell times during which the drug remains in the kidney, and (b) uneven distribution of the drug within the kidney. By way of further example, while the transurethral retrograde approach may allow for more consistent or homogenous distribution of the drug in the kidney, it requires (general anesthetic), (b) a new placement of a catheter under general anesthetic each time a patient requires a further delivery of drug, and (c) does not provide a mechanism for increasing time.

The present systems address a number of these shortcomings. For example, the present systems allow repeated access to the kidneys over time, without incisions and without repeated administration of general anesthetic. Additionally, the present systems allow for homogeneous distribution of a drug within a kidney and the maintenance of the drug within a kidney for an extended period of time. The present systems also follow a familiar transurethral retrograde approach through the urethra and ureter along a path that will already be familiar to urologists and interventional radiologists, and can be used with patients on blood thinners since incisions are not necessary for deployment. Additionally, the structure of the present systems can be formed entirely of known (and other) materials that are biocompatible according to the ISO 10993 standard.

In some configurations of the present systems, the system comprises a urethral catheter and a ureteral catheter. In some such configurations, the urethral catheter comprises an elongated urethral catheter body having a proximal end, a distal end, and a first length extending from the proximal end and the distal end, the urethral catheter having a urethral catheter balloon coupled to the urethral catheter body nearer the distal end than the proximal end, and the urethral catheter body defines: a primary lumen extending through the body to a distal port in an outer surface of the body at a position that is more distal than the balloon; and a secondary lumen extending through the body and in fluid communication with an interior of the urethral catheter balloon. In some such configurations, the ureteral catheter comprises an elongated ureteral catheter body having a proximal end, a distal end, and a second length extending from the proximal end and the distal end, the second length being longer than the first length, the ureteral catheter having a ureteral catheter balloon coupled to the ureteral catheter body nearer the distal end than the proximal end, and the ureteral catheter body defines: a primary lumen extending through the body to one or more distal ports in an outer surface of the body at a position that is more distal than the balloon; and a secondary lumen extending through the body and in fluid communication with an interior of the ureteral catheter balloon. In such configurations, the urethral catheter is configured to extend into a patient's urethra such that the urethral catheter balloon is disposed in the patient's bladder, and the ureteral catheter is configured to extend over a guidewire and through the primary lumen of the urethral catheter body into the patient's ureter such that the ureteral catheter balloon is disposed in the ureter or a corresponding one of the patient's kidneys.

In some of the foregoing configurations of the present systems, the ureteral catheter body includes a J-tip extending from the distal end of the body toward the proximal end, where the J-tip is biased toward a curved shape. In some such configurations, the entirety of the J-tip is distal of the ureteral catheter balloon.

In some of the foregoing configurations of the present systems, the one or more distal ports of the ureteral catheter body comprise a plurality of distal ports spanning a first longitudinal extent of at least fifteen (15) millimeters (mm). In some such configurations, the first longitudinal extent has a length of 20 mm to 60 mm.

In some of the foregoing configurations of the present systems, the plurality of first ports are spaced from one another along the first longitudinal extent.

In some of the foregoing configurations of the present systems, the second length of the ureteral catheter body is between 300 mm and 500 mm.

In some of the foregoing configurations of the present systems, the urethral catheter includes a reinforced tip at the distal end of the urethral catheter body, and where the distal port of the urethral catheter body extends through the distal end of the urethral catheter body.

In some of the foregoing configurations of the present systems, the urethral catheter body has a nominal diameter of from 10 French (Fr) to 18 Fr.

In some of the foregoing configurations of the present systems, the ureteral catheter body has a nominal diameter of from 4 Fr to 9 Fr.

In some of the foregoing configurations of the present systems, the proximal end of the urethral catheter body is configured to be coupled to the ureteral catheter body to resist movement of the ureteral catheter body relative to the urethral catheter body. In some such configurations, the urethral catheter body defines an opening configured to receive a suture to couple the ureteral catheter body to the urethral catheter body.

In some of the foregoing configurations of the present systems, the system further comprises: a guidewire having a proximal end, a distal end, and a third length extending from the proximal end and the distal end, the guidewire configured to extend through the primary lumen of the urethral catheter, through the primary lumen of the ureteral catheter, or through the primary lumens of both of the urethral and ureteral catheters, where the third length is greater than the second length.

In some of the foregoing configurations of the present systems, the urethral catheter further comprises a Luer fitting in fluid communication with the secondary lumen of the urethral catheter body.

In some of the foregoing configurations of the present systems, the ureteral catheter further comprises a Luer fitting in fluid communication with the secondary lumen of the ureteral catheter body.

In some implementations of the present methods, the method comprises: delivering a drug into a kidney of a patient using one of the foregoing configurations of the present systems, while: the urethral catheter body extends through the urethra of the patient and the urethral catheter balloon is at least partially inflated within the bladder of the patient; and the ureteral catheter extends through the primary lumen of the urethral catheter body and through a ureter of the patient with the ureteral catheter balloon at least partially inflated within the ureter or a corresponding kidney of the patient; where the drug is delivered into the kidney through the primary lumen of the ureteral catheter body.

In some of the foregoing implementations of the present methods, the method further comprises: after delivering the drug, plugging a proximal end of the primary lumen of the ureteral catheter body to retain the drug in the kidney. In some such implementations, the method further comprises: unplugging the proximal end of the primary lumen of the ureteral catheter body to allow the drug to drain out of the kidney. In some of such configurations, the method further comprises: coupling a urine bag to the urethral catheter body with an interior of the urine bag in fluid communication with the primary lumen of the urethral catheter body. In some such configurations, the urine bag is coupled to the urethral catheter body while the ureteral catheter remains in the primary lumen of the urethral catheter body.

In some of the foregoing implementations of the present methods, the method further comprises, prior to delivering the drug: inserting a guidewire through the urethra and the ureter of the patient such that a distal end of the guidewire extends into the corresponding kidney of the patient; inserting the urethral catheter over the guidewire and through the patient's urethra such that the balloon is disposed in the patient's bladder; at least partially inflating the urethral catheter balloon in the patient's bladder; inserting the ureteral catheter body over the guidewire, through the primary lumen of the urethral catheter body, and through the patient's ureter such that the ureteral catheter balloon is disposed in the corresponding kidney of the patient; and at least partially inflating the ureteral catheter balloon in the patient's kidney; and removing the guidewire from the primary lumen of the urethral catheter body.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of-rather than comprise/include/contain/have-any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

Some details associated with the aspects of the present disclosure are described above, and others are described below. Other implementations, advantages, and features of the present disclosure will become apparent after review of the entire application, including the Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical labels or reference numbers do not necessarily indicate an identical structure. Rather, the same reference number may be used to indicate a similar feature or a feature with similar functionality, as may non-identical reference numbers. Dimensioned figures are drawn to scale (unless otherwise noted), meaning the sizes of the depicted elements are accurate relative to each other for at least the embodiment depicted in the figures.

FIG. 1 depicts a side view of a first example of the present catheter systems.

FIG. 2A depicts an enlarged, cutaway side view of a proximal portion of the catheter system of FIG. 1.

FIG. 2B depicts an enlarged, cutaway side view of a distal portion of the catheter system of FIG. 1.

FIG. 3 depicts a side view of a urethral catheter of the catheter system of FIG. 1.

FIG. 4 depicts a side view of a ureteral catheter of the catheter system of FIG. 1.

FIG. 5 depicts a schematic cross-sectional diagram showing a representative configuration of a catheter with a larger primary lumen and a smaller secondary lumen.

FIG. 6 depicts a schematic diagram of the catheter system of FIG. 1 deployed in a urinary tract of a patient.

### DETAILED DESCRIPTION OF ILLUSTRATIVE EXAMPLES

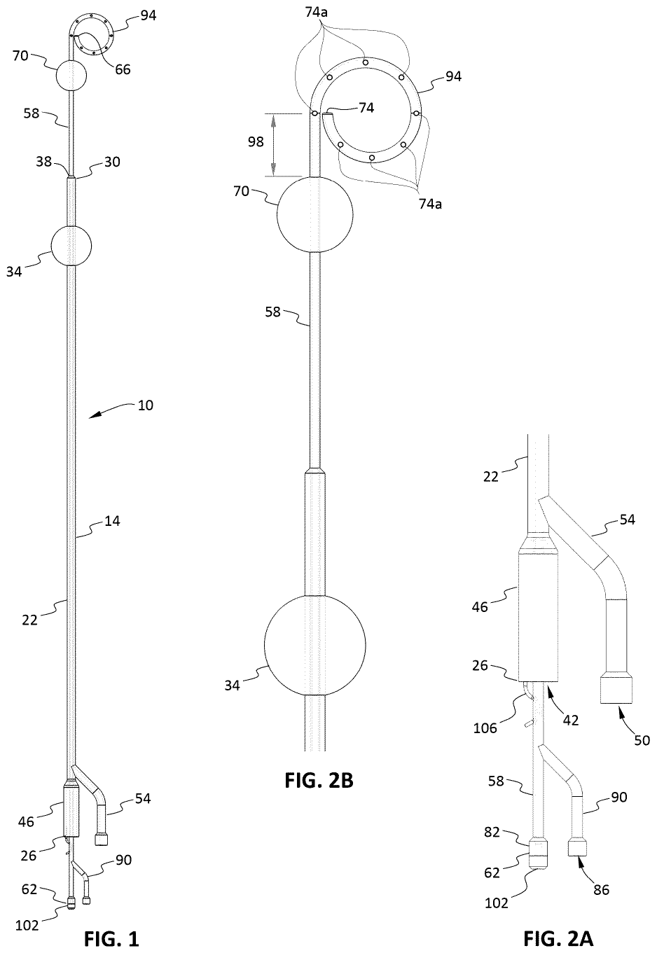

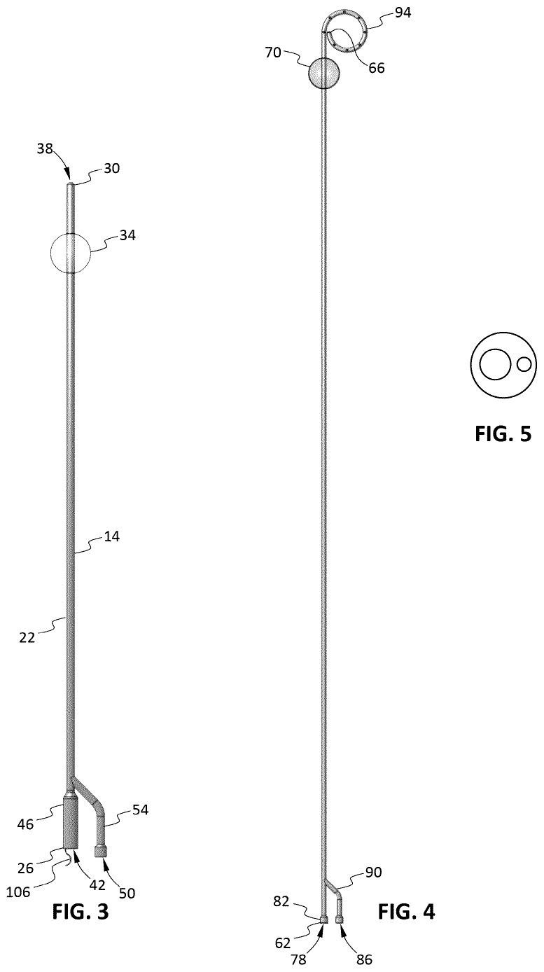

Referring now to the drawings and more particularly to FIGS. 1, 2A, 2B, 3, 4, and 5, shown there and designated by the reference numeral 10 is a first example of the present catheter systems. As shown, system 10 comprises a urethral catheter 14 and a ureteral catheter 18. In the depicted example, urethral catheter 14 comprises an elongated urethral catheter body 22 having a proximal end 26, a distal end 30, and a first length extending from proximal end 26 to distal end 30 (along the length of body 22, e.g., along a straight line when the distal portion of body 22 is straightened). As also shown, urethral catheter 14 has a urethral catheter balloon 34 coupled to body 22 nearer distal end 30 than proximal end 26. In this example, balloon 34 has a fully inflated outer diameter of 20 mm; in other configurations, balloon 34 can have a diameter of between any two of: 10, 15, 20, 25, and 30 mm.

Urethral catheter body 22 also defines: a primary lumen extending through the body to a distal port 38 in an outer surface of body 22 at a position that is more distal than balloon 34 (e.g., through distal end 30), and a secondary lumen extending through body 22 and in fluid communication with an interior of urethral catheter balloon 34. In the depicted example, the primary lumen extends from a proximal opening 42 in a hub or connector portion (e.g., funnel end) 46 at proximal end 26, longitudinally through the length of body 22, and through distal port 38. In configurations, such as the one shown, hub or connector portion 46 is configured to be coupled to a urine bag. The second lumen extends from a proximal opening or balloon port 50 in a proximal end of a connector tail 54 (e.g., with a Luer fitting at its proximal end) that extends from the main portion of urethral catheter body 22. The second lumen then extends through connector tail 54, through a portion of the length of body 22, and through an opening in an outer surface of body 22 within balloon 34. Urethral catheter 14 may, for example, comprise a Councill-tip urethral catheter that includes a reinforced tip at distal end 30, where distal port 38 extends through distal end 30, such as to permit a guidewire to pass through the primary lumen of body 22 and through distal end 30.

In the depicted example, ureteral catheter 18 comprises an elongated ureteral catheter body 58 having a proximal end 62, a distal end 66, and a second length extending from proximal end 62 to distal end 66 (along the length of body 58, e.g., along a straight line when the distal portion of body 58 is straightened). As shown, ureteral catheter body 58 is sized to fit within and through the primary lumen of urethral catheter body 22 (e.g., ureteral catheter body 58 has an outer diameter that is smaller than an inner diameter of the primary lumen of urethral catheter body 22). By way of example, in the depicted configuration, urethral catheter body 22 has a nominal outer diameter of 4 mm or 12 French (Fr), and ureteral catheter body 58 has a nominal outer diameter of 2 mm or 6 Fr. In other configurations, urethral catheter body 22 has a nominal outer diameter of from 10 Fr to 18 Fr (3.33 mm to 6 mm), and ureteral catheter body 58 has a nominal outer diameter of from 4 Fr to 9 Fr (1.33 mm to 3 mm).

As also shown, ureteral catheter 18 has a ureteral catheter balloon 70 coupled to body 58 nearer distal end 66 than proximal end 62. In this example, balloon 70 has a fully inflated outer diameter of 15 mm; in other configurations, balloon 70 can have a diameter of between any two of: 10, 15, 20, and 25 mm.

Ureteral catheter body 58 also defines: a primary lumen extending through the body to one or more distal ports 74 in an outer surface of body 58 at a position that is more distal than balloon 70 (e.g., through distal end 66), and a secondary lumen extending through body 58 and in fluid communication with an interior of ureteral catheter balloon 70. In the depicted example, the primary lumen extends from a proximal opening 78 in a hub or connector portion (e.g., Luer fitting) 82 at proximal end 62, longitudinally through the length of body 58, and through distal port 74. The second lumen extends from a proximal opening or balloon port 86 in a proximal end of a connector tail 90 (e.g., with a Luer fitting at its proximal end) that extends from the main portion of ureteral catheter body 58. The second lumen then extends through connector tail 90, through a portion of the length of body 58, and through an opening in an outer surface of body 58 within balloon 70.

FIG. 5 shows an example of a cross-section of a catheter body having a larger primary lumen and a smaller secondary lumen. While the depicted lumens have circular cross-sectional shapes, any of various shapes of the lumens may be used as is known in the art. For the urethral catheter body 18 shown, the primary lumen will extend from proximal opening 42 to distal opening 38, and the secondary lumen will extend from proximal opening 50 in connector tail 54 to a port within balloon 34 (not proximally beyond connector tail 54 and not distally beyond balloon 34). For the ureteral catheter body 58 shown, the primary lumen will extend from proximal opening 78 to distal end 66 (but may not necessarily extend through distal end 66), and the secondary lumen will extend from proximal opening 86 in connector tail 90 to a port within balloon 70 (not proximally beyond connector tail 90 and not distally beyond balloon 70). In some configurations, such as the one shown in FIGS. 1-4, the primary lumen of ureteral catheter body 58 extends through distal end 66 such that a guidewire can extend the length of the primary lumen and through distal end 66 to enable ureteral catheter body 58 to be advanced over a guidewire to facilitate deployment of ureteral catheter body 58 into a patient's kidney as described below.

As can be seen especially in FIG. 1, the second length of ureteral catheter body 58 is greater than the first length of urethral catheter body 22, such that when ureteral catheter body 58 is inserted through the primary lumen of urethral catheter body 22, distal end 66 can extend distally beyond distal end 30 of urethral catheter body 22. For example, the second length of ureteral catheter body 58 is in some configurations from 300 mm to 500 mm, and may differ between configurations for different categories of patients (e.g., 350 mm for women, 450 mm for men, 300 mm for children).

As described in more detail below, in the depicted example of system 10, urethral catheter 14 is configured to extend into a patient's urethra such that the urethral catheter balloon 34 is disposed in the patient's bladder, and ureteral catheter 18 is configured to extend over a guidewire and through the primary lumen of urethral catheter body 22 into the patient's ureter such that ureteral catheter balloon 70 is disposed in the ureter or a corresponding one of the patient's kidneys.

In the depicted example, a distal portion of ureteral catheter body 58 is shaped to facilitate distributed delivery of fluid to multiple parts of a patient's kidney. For example, as shown, ureteral catheter body 58 includes a J-tip 94 extending from distal end 66 of body 58 toward proximal end 62 (e.g., from distal end 66 toward balloon 70). As shown, J-tip 94 is biased toward a curved shape (e.g., with a radius of greater than any one of, or between any two of: 8, 10, 12, 14, 16, 18, or 20 millimeters (mm)). In the depicted example, the curved shape circumferentially spans a full 360 degrees; in other configurations, the curved shape can span at least any one of, or between any two of, 45, 90, 135, 180, 225, 270, 315, or 360 degrees. Additionally, in this example, the entirety of J-tip 94 is distal of ureteral catheter balloon 70, with the curve of J-tip 94 begins distal of balloon 70 by a distance 98, which may for example be at least any one or between any two of: 10, 15, 20, 25, or 30 mm. While J-tip 94 is biased toward a curved configuration, such as the one shown, J-tip is flexible enough to be substantially straightened to allow ureteral catheter body 58 to be advanced over a guidewire, such as for deployment inside a patient as described below.

Additionally, to further facilitate distributed delivery of fluid to multiple parts of a patient's kidney, the depicted example of ureteral catheter body 58 defines a plurality of additional distal ports 74a extending through a peripheral surface of body 58 and in fluid communication with the primary lumen of the ureteral catheter body 58. As shown, distal ports 74a are spaced from one another (e.g., at equal intervals) along a longitudinal extent along body 58 from a proximal-most port 74a to a distal-most port 74a, which longitudinal extent may be at least 15 mm in length (e.g., 20 mm to 60 mm, or greater).

In some configurations of system 10, such as the one shown, proximal end 26 of urethral catheter body 22 is configured to be coupled to ureteral catheter body 58 to resist movement (e.g., unintended withdrawal) of ureteral catheter body 58 relative to urethral catheter body 22. For example, in the depicted example, urethral catheter body 22 defines an opening configured to receive a suture 102 to couple ureteral catheter body 58 to urethral catheter body 22. For example, upon insertion of ureteral catheter body 58 in the primary lumen of urethral catheter body 22, suture 102 can be wrapped around, tied, and/or otherwise coupled to ureteral catheter body 58 to resist movement of ureteral catheter body 58 relative to urethral catheter body 22.

In the depicted configuration, ureteral catheter 18 also includes a plug 106 configured to be inserted into proximal port 78 to prevent liquid from exiting proximal port 78, for example to increase the duration of time that a drug is resident in a patient's kidney, as described further below.

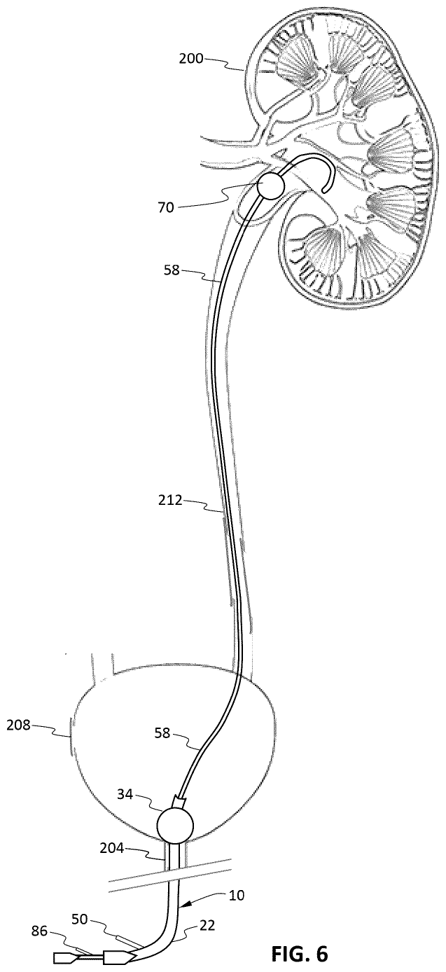

Referring now to FIG. 6, the present systems can be used to deliver one or more drugs into a kidney of a patient. In some implementations, the present methods comprise: delivering a drug into a kidney 200 of a patient using one of the present systems (e.g., 10), while: the urethral catheter body (22) extends through the urethra 204 of the patient and the urethral catheter balloon (34) is at least partially inflated within a bladder 208 of the patient; and the ureteral catheter body (58) extends through the primary lumen of the urethral catheter body (22) and through a ureter 212 of the patient with the ureteral catheter balloon (70) at least partially inflated within the ureter 212 or a corresponding kidney 200 of the patient. In such implementations, the drug is delivered into the kidney 200 through the primary lumen (e.g., via proximal opening 42) of the ureteral catheter body (58).

In some of the foregoing implementations of the present methods, after the drug is delivered, the method further comprises plugging (e.g., with plug 102) a proximal end 62 (e.g., proximal port 78) of the primary lumen of the ureteral catheter body to retain the drug in the kidney. After a desired duration or dwell time of the drug being in the kidney, proximal end 62 of ureteral catheter body 58 can be unplugged to allow the drug to drain out of the kidney. For patients who are likely to need further treatments, the present catheter systems (e.g., 10) can remain in the patient (e.g., for a period of multiple days, 1 week, 2 weeks, or more), and a urine bag can be coupled to proximal end 26 of the urethral catheter (e.g., while ureteral catheter body 58 remains in the primary lumen of urethral catheter body 22 and/or after re-plugging the proximal end of the ureteral catheter body 22).

Some of the present methods comprise (e.g., prior to delivering a drug): inserting a guidewire through the urethra (204) and the ureter (212) of the patient such that a distal end of the guidewire extends into the corresponding kidney (200) of the patient; inserting the urethral catheter body (22) over the guidewire and through the patient's urethra (204) such that the urethral catheter balloon (34) is disposed in the patient's bladder (208); at least partially inflating the urethral catheter balloon (34) in the patient's bladder, for example via a balloon port (50); inserting ureteral catheter body (58) over the guidewire, through the primary lumen of the urethral catheter body (22), and through the patient's ureter (212) such that the ureteral catheter balloon (70) is disposed in the corresponding kidney (200) of the patient; at least partially inflating the ureteral catheter balloon (70) in the patient's kidney, for example a via balloon port (86); and removing the guidewire from the primary lumen of the urethral catheter body (22).

In one more-detailed example of the present methods of deploying the present catheter systems and the delivery of a drug to a patient's kidney, the method comprises the following steps:
The intrarenal volume/amount of fluid (e.g., drug) needed to fill the inside of the kidney is first measured. Under fluoroscopy, a conventional ureteral catheter is advanced in the kidney and contrast agent is injected to fill the inside of the kidney and the volume needed to fill the inside of the kidney is recorded.
Next, one of the present catheter systems is deployed, as follows:
   Under fluoroscopy, a guidewire is placed in the kidney through a cystoscope.
   The cystoscope is then removed, the guidewire kept in place in the kidney.
   Urethral catheter body 22 (e.g., of a Councill-tip urethral catheter) is then advanced over the guidewire and into the bladder (208), and the urethral catheter balloon (34) is inflated with a desired volume (e.g., 5 mL) of sterile water or saline while the balloon is in the patient's bladder.
   Ureteral catheter body 58 is then advanced over the guidewire and through the primary lumen of the urethral catheter body 22, and the ureteral catheter balloon (70) is inflated with a desired volume (e.g., 2-3 mL) of sterile water or saline while the balloon (70) is in an upper portion of the patient's ureter (212) or kidney (200) for example at the ureteropelvic junction.
   Once the ureteral catheter body (58) is at the desired location, the ureteral catheter body (58) can be coupled to the urethral catheter body (22), such as via a suture (106) or other structure or connection sufficient to resist undesirable movement of the urethral catheter body (22) relative to the ureteral catheter body (58).
   With the distal port(s) (e.g., 74, 74a) of the ureteral catheter body (58) positioned in the patient's kidney, the drug can be administered in a volume sufficient to fill the interior of the kidney (as determined in the first part of the method) and to fill the primary lumen of the ureteral catheter body (58).
   With the kidney 200 and the primary lumen of the ureteral catheter body (58) filled with the drug, the proximal port of the ureteral catheter body (58) can be plugged or closed (e.g., with plug 102) to maintain the drug in the kidney 200 and ensure contact between the drug and the interior of the kidney 200 for a desired period of time, such as 1 hour, 2 hours, or more).
   After the desired dwell time has passed, the drug can be drained from the kidney 200 through the ureteral catheter body (58), such as by removing the plug (102) from the proximal end of the ureteral catheter body (58).
   If the patient is not expected to need further treatment, the balloons (34, 70) can be deflated, and the urethral and ureteral catheters removed from the patient.
   If instead the patient is expected to need further treatment, a urine collection bag can be connected to the proximal end (26) of the urethral catheter body (22), such as after re-inserting the plug (102) into the proximal end (62) of the ureteral catheter body 22), and the patient can be discharged to later return for further drug administrations via the catheter system (e.g., 10).

In another more-detailed example of the present methods of replacing the present catheter systems in a patient's kidney, the method comprises the following steps:
Under fluoroscopy, a guidewire can be advanced through the primary lumen of the ureteral catheter body (58) into the patient's kidney.
The ureteral catheter balloon (70) can then be deflated, and structure (e.g., suture 106) coupling the ureteral catheter body (58) to the urethral catheter body (22) can be cut or otherwise uncoupled, to allow the ureteral catheter body (58) to be removed over the guidewire while maintaining the guidewire in place.
The urethral catheter balloon (34) can then be deflated to allow the urethral catheter body (22) to be removed over the guidewire while maintaining the guidewire in place.
A new urethral catheter body (22) (e.g., of a Councill-tip urethral catheter) can then be advanced over the guidewire and into the bladder (208), and the urethral catheter balloon (34) inflated with a desired volume (e.g., 5 mL) of sterile water or saline while the balloon is in the patient's bladder.
A new ureteral catheter body (58) can then be advanced over the guidewire and through the primary lumen of the urethral catheter body 22, and the ureteral catheter balloon (70) inflated with a desired volume (e.g., 2-3 mL) of sterile water or saline while the balloon (70) is in an upper portion of the patient's ureter (212) or kidney (200) for example at the ureteropelvic junction.
Once the new ureteral catheter body (58) is at the desired location, the ureteral catheter body (58) can be coupled to the urethral catheter body (22), such as via a suture (106) or other structure or connection sufficient to resist undesirable movement of the urethral catheter body (22) relative to the ureteral catheter body (58).
The guidewire can then be removed through the primary lumens of the urethral and ureteral catheters, after which a drug can be administered.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A system comprising:
a urethral catheter comprising an elongated urethral catheter body having a proximal end, a distal end, and a first length extending from the proximal end and the distal end, the urethral catheter having a urethral catheter balloon coupled to the urethral catheter body nearer the distal end than the proximal end, the urethral catheter body defining:
a primary lumen extending through the body to a distal port in an outer surface of the body at a position that is more distal than the balloon; and
a secondary lumen extending through the body and in fluid communication with an interior of the urethral catheter balloon; and
a ureteral catheter comprising an elongated ureteral catheter body having a proximal end, a distal end, and a second length extending from the proximal end and the distal end, the second length being longer than the first length, the ureteral catheter having a ureteral catheter balloon coupled to the ureteral catheter body nearer the distal end than the proximal end, the ureteral catheter body defining:
a primary lumen extending through the body to one or more distal ports in an outer surface of the body at a position that is more distal than the balloon; and
a secondary lumen extending through the body and in fluid communication with an interior of the ureteral catheter balloon; and
where the urethral catheter is configured to extend into a patient's urethra such that the urethral catheter balloon is disposed in the patient's bladder, and the ureteral catheter is configured to extend over a guidewire and through the primary lumen of the urethral catheter body into the patient's ureter such that the ureteral catheter balloon is disposed in the ureter or a corresponding one of the patient's kidneys.

2. The system of claim 1, where the ureteral catheter body includes a J-tip extending from the distal end of the body toward the proximal end, where the J-tip is biased toward a curved shape.

3. The system of claim 2, where the entirety of the J-tip is distal of the ureteral catheter balloon.

4. The system any claim 1, where the one or more distal ports of the ureteral catheter body comprise a plurality of distal ports spanning a first longitudinal extent of at least fifteen (15) millimeters (mm).

5. The system of claim 4, where the first longitudinal extent has a length of 20 mm to 60 mm.

6. The system of claim 1, where the plurality of first ports are spaced from one another along the first longitudinal extent.

7. The system of claim 1, where the second length of the ureteral catheter body is between 300 mm and 500 mm.

8. The system of claim 1, where the urethral catheter includes a reinforced tip at the distal end of the urethral catheter body, and where the distal port of the urethral catheter body extends through the distal end of the urethral catheter body.

9. The system of claim 1, where the urethral catheter body has a nominal diameter of from 10 French (Fr) to 18 Fr or of from 4 Fr to 9 Fr.

10. (canceled)

11. The system of claim 1, where the proximal end of the urethral catheter body is configured to be coupled to the ureteral catheter body to resist movement of the ureteral catheter body relative to the urethral catheter body.

12. The system of claim 11, where the urethral catheter body defines an opening configured to receive a suture to couple the ureteral catheter body to the urethral catheter body.

13. The system of claim 1, further comprising:
a guidewire having a proximal end, a distal end, and a third length extending from the proximal end and the distal end, the guidewire configured to extend through the primary lumen of the urethral catheter, through the primary lumen of the ureteral catheter, or through the primary lumens of both of the urethral and ureteral catheters, where the third length is greater than the second length.

14. The system of claim 1, where the urethral catheter further comprises a Luer fitting in fluid communication with the secondary lumen of the urethral catheter body.

15. The system of claim 1, where the ureteral catheter further comprises a Luer fitting in fluid communication with the secondary lumen of the ureteral catheter body.

16. A method comprising:
delivering a drug into a kidney of a patient using the system of claim 1, while:
the urethral catheter body extends through the urethra of the patient and the urethral catheter balloon is at least partially inflated within the bladder of the patient; and
the ureteral catheter extends through the primary lumen of the urethral catheter body and through a ureter of the patient with the ureteral catheter balloon at least partially inflated within the ureter or a corresponding kidney of the patient;
where the drug is delivered into the kidney through the primary lumen of the ureteral catheter body.

17. The method of claim 16, further comprising:
after delivering the drug, plugging a proximal end of the primary lumen of the ureteral catheter body to retain the drug in the kidney.

18. The method of claim 17, further comprising:
unplugging the proximal end of the primary lumen of the ureteral catheter body to allow the drug to drain out of the kidney.

19. The method of claim 18, further comprising:
coupling a urine bag to the urethral catheter body with an interior of the urine bag in fluid communication with the primary lumen of the urethral catheter body.

20. The method of claim 19, where the urine bag is coupled to the urethral catheter body while the ureteral catheter remains in the primary lumen of the urethral catheter body.

21. The method of claim 16, further comprising, prior to delivering the drug:
inserting a guidewire through the urethra and the ureter of the patient such that a distal end of the guidewire extends into the corresponding kidney of the patient;
inserting the urethral catheter over the guidewire and through the patient's urethra such that the balloon is disposed in the patient's bladder;
at least partially inflating the urethral catheter balloon in the patient's bladder;
inserting the ureteral catheter body over the guidewire, through the primary lumen of the urethral catheter body, and through the patient's ureter such that the ureteral catheter balloon is disposed in the corresponding kidney of the patient;
at least partially inflating the ureteral catheter balloon in the patient's kidney; and removing the guidewire from the primary lumen of the urethral catheter body.
